(19) United States
(12) Patent Application Publication
(10) Pub. No.: US 2023/0364009 A1
(43) Pub. Date: Nov. 16, 2023

(54) Implantable Medical Device for the Delivery of an Antipsychotic

(71) Applicant: Celanese EVA Performance Polymers LLC, Irving, TX (US)

(72) Inventors: Jeffrey Haley, Norwood, OH (US); Anthony Palmieri, Hillsborough, NJ (US)

(21) Appl. No.: 18/315,590

(22) Filed: May 11, 2023

Related U.S. Application Data
(60) Provisional application No. 63/341,103, filed on May 12, 2022.

(51) Int. Cl. A61K 9/00; A61K 31/519; B29C 48/88; B29C 48/68; B29C 48/00; B29K 23/00; B29L 31/00

(52) U.S. Cl. CPC A61K 9/0036; A61K 31/519; B29C 48/911; B29C 48/68; B29C 48/0022; B29C 48/022; B29K 2023/083; B29L 2031/753

(57) ABSTRACT

An implantable medical device is provided. The core includes a core polymer matrix within which is dispersed a therapeutic agent comprising one or more antipsychotics. The core polymer matrix contains an ethylene vinyl acetate copolymer. The ethylene vinyl acetate copolymer has a melt flow index of about 1 to about 400 grams per 10 minutes as determined in accordance with ASTM D1238-20 at a temperature of 190° C. and a load of 2.16 kilograms.

### RELATED APPLICATION

The present application is based upon and claims priority to U.S. Provisional Patent Application Ser. No. 63/341,103, having a filing date of May 12, 2022, which is incorporated herein by reference.

### BACKGROUND

Antipsychotics, including atypical antipsychotics, are used to treat conditions such as schizophrenia, major depressive disorder, bipolar disorder, and schizoaffective disorder. The goals of treatment with antipsychotic medication are to reduce or eliminate symptoms and to improve patient functioning over the long term. However, poor medication adherence contributes to negative treatment response, symptom relapse, and hospitalization. Relapse rates are up to five times higher in patients who discontinue their antipsychotic medication compared to those who continue with treatment. Even brief gaps in oral antipsychotic medication have been shown to increase the risk of relapse and hospitalization.

As such, a need continues to exist for an implantable delivery device that is capable of delivering one or more antipsychotics over a sustained period of time.

### BRIEF SUMMARY

In accordance with one embodiment of the present disclosure, an implantable medical device is disclosed. The device includes a core containing a core polymer matrix having one or more therapeutic agents including one or more antipsychotics dispersed therein. The core polymer matrix contains an ethylene vinyl acetate copolymer. The ethylene vinyl acetate copolymer has a has a melt flow index of about 1 to about 400 grams per 10 minutes as determined in accordance with ASTM D1238-20 at a temperature of 190° C. and a load of 2.16 kilograms.

In accordance with another embodiment of the present disclosure, a method for prohibiting and/or treating a condition, disease, and/or cosmetic state of a patient is provided. The method includes subcutaneously implanting a device including a core containing a core polymer matrix having one or more therapeutic agents including one or more antipsychotics dispersed therein. The core polymer matrix contains an ethylene vinyl acetate copolymer. The ethylene vinyl acetate copolymer has a has a melt flow index of about 1 to about 400 grams per 10 minutes as determined in accordance with ASTM D1238-20 at a temperature of 190° C. and a load of 2.16 kilograms.

In accordance with another embodiment of the present disclosure, a method for prohibiting and/or treating a condition, disease, and/or cosmetic state of a patient is provided. The method includes intravaginally inserting a device including a core containing a core polymer matrix having one or more therapeutic agents including one or more antipsychotics dispersed therein. The core polymer matrix contains an ethylene vinyl acetate copolymer. The ethylene vinyl acetate copolymer has a has a melt flow index of about 1 to about 400 grams per 10 minutes as determined in accordance with ASTM D1238-20 at a temperature of 190° C. and a load of 2.16 kilograms.

In accordance with another embodiment of the present disclosure provided is a method for inhibiting D₂ dopaminergic receptors and 5-HT_{2A} serotonergic receptors in the brain, comprising subcutaneously inserting one or more implantable devices in a patient, the implantable device comprising a core comprising a core polymer matrix within which is dispersed a therapeutic agent comprising one or more antipsychotics, the core polymer matrix containing an ethylene vinyl acetate copolymer, wherein the ethylene vinyl acetate copolymer has a vinyl acetate content of from about 10 wt. % to about 60 wt. % and/or a melting temperature of from about 40° C. to about 120° C. as determined in accordance with ASTM D3418-15.

In accordance with another embodiment of the present disclosure, provided is a method of manufacturing an implantable device. The method includes melt-blending a core polymer matrix containing an ethylene vinyl acetate copolymer and a therapeutic agent comprising one or more antipsychotics in an extruder barrel at a first temperature, the ethylene vinyl acetate copolymer having a melt flow index of from about 1 to about 400 grams per 10 minutes as determined in accordance with ASTM D1238-20 at a temperature of 190° C. and a load of 2.16 kilograms; mixing the core polymer matrix and therapeutic agent in the extruder barrel at a second temperature to form a mixture of core polymer matrix and therapeutic agent; extruding the mixture of core polymer matrix and therapeutic agent from the extruder barrel forming a core of the implantable device; cooling the core; and cutting the core to form the implantable device.

Other features and aspects of the present disclosure are set forth in greater detail below.

### BRIEF DESCRIPTION OF THE DRAWINGS

A full and enabling disclosure of the present disclosure, including the best mode thereof, directed to one of ordinary skill in the art, is set forth more particularly in the remainder of the specification, which makes reference to the appended drawings in which:

FIG. 1 is a perspective view of one embodiment of the implantable medical device of the present disclosure;

FIG. 2 is a cross-sectional view of the implantable medical device of FIG. 1;

FIG. 3 is a perspective view of one embodiment of the implantable medical device of the present disclosure;

FIG. 4 is a cross-sectional view of the implantable medical device of FIG. 3;

FIG. 5 is a perspective view of one embodiment of the implantable medical device of the present disclosure;

FIG. 6 is a perspective view of another embodiment of the implantable medical device of the present disclosure;

FIG. 7 is a cross-sectional view of the implantable medical device of FIG. 6;

FIG. 8 is a cross-sectional view of an implantable medical device, specifically a vaginal ring, of the present disclosure;

FIG. 9 is a cross-sectional view of an implantable medical device, specifically a vaginal ring, of the present disclosure;

FIG. 10 is a cross-sectional view of an implantable medical device, specifically a vaginal ring, of the present disclosure;

FIG. 11 is a graph showing the percent release of risperidone for Example 1-4 as referenced above in Table 1;

FIG. 12 is a graph showing the cumulative release of risperidone per surface area versus time for Examples 1-4; and

FIG. 13 is a graph showing the cumulative release of risperidone for Examples 4-7.

Repeat use of references characters in the present specification and drawing is intended to represent same or analogous features or elements of the disclosure.

### DETAILED DESCRIPTION

It is to be understood by one of ordinary skill in the art that the present discussion is a description of exemplary embodiments only and is not intended as limiting the broader aspects of the present disclosure.

Generally speaking, the present disclosure is directed to an implantable medical device that is capable of delivering an antipsychotic to a patient (e.g., human, pet, farm animal, racehorse, etc.) over a sustained period of time to help prohibit and/or treat a condition, disease, and/or cosmetic state of the patient. The condition and/or disease can include schizophrenia, bipolar disorder, major depressive disorder, or schizoaffective disorder. The implantable medical device includes a core containing a core polymer matrix containing an ethylene vinyl acetate copolymer having one or more therapeutic agents dispersed therein. The therapeutic agent includes one or more antipsychotics. The ethylene vinyl acetate copolymer has a vinyl acetate content of from about 10 wt. % to about 60 wt. % and/or a melting temperature of from about 40° C. to about 120° C. as determined in accordance with ASTM D3418-15.

Various embodiments of the present disclosure will now be described in more detail.

### I. Core

As indicated above, the core polymer matrix contains at least a polymer that is generally hydrophobic in nature so that it can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the core polymer matrix. Typically, the melt flow index of the hydrophobic polymer ranges from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-13 at a temperature of 190° C. and a load of 2.16 kilograms.

In certain embodiments, the core polymer matrix may contain a semi-crystalline olefin copolymer. The melting temperature of such an olefin copolymer may, for instance, range from about 40° C. to about 140° C., in some embodiments from about 50° C. to about 125° C., and in some embodiments, from about 60° C. to about 120° C., as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, certain aspects of the copolymer can be selectively controlled to help achieve the desired release properties. For instance, the polar monomeric content of the copolymer may be selectively controlled to be within a range of from about 10 wt. % to about 60 wt. %, in some embodiments about 20 wt. % to about 60 wt. %, and in some embodiments, from about 25 wt. % to about 50 wt. %. Conversely, the olefin monomeric content of the copolymer may likewise be within a range of from about 40 wt. % to about 90 wt. %, in some embodiments about 40 wt. % to about 80 wt. %, and in some embodiments, from about 50 wt. % to about 75 wt. %.

In one particular embodiment, for example, the core polymer matrix may contain at least one ethylene vinyl acetate polymer, which is a copolymer that is derived from at least one ethylene monomer and at least one vinyl acetate monomer. In certain cases, the present inventors have discovered that certain aspects of the copolymer can be selectively controlled to help achieve the desired release properties. For instance, the vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. In certain embodiments, the vinyl acetate content ranges from about 25 wt. % to about 32 wt. %. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments from about 1 to about 200 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55). In embodiments, the ethylene vinyl acetate copolymer in the core polymer matrix is from about 20 wt. % to about 90 wt. %, such as from about 30 wt. % to about 80 wt. %, such as from about 40 wt. % to about 70 wt. %.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain embodiments, it may also be desirable to employ blends of an ethylene vinyl acetate copolymer and another hydrophobic polymer such that the overall blend and polymer matrix have a melting temperature and/or melt flow index within the range noted above. For example, the polymer matrix may contain a first ethylene vinyl acetate copolymer and a second ethylene vinyl acetate copolymer having a melting temperature that is greater than the melting temperature of the first copolymer. The second copolymer may likewise have a melt flow index that is the same, lower, or higher than the corresponding melt flow index of the first copolymer. The first copolymer may, for instance, have a melting temperature of from about 20° C. to about 60° C., in some embodiments from about 25° C. to about 55° C., and in some embodiments, from about 30° C. to about 50° C., such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second copolymer may likewise have a melting temperature of from about 50° C. to about 100° C., in some embodiments from about 55° C. to about 90° C., and in some embodiments, from about 60° C. to about 80° C., such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first copolymer may constitute from about 20 wt. % to about 80 wt. %, in some embodiments from about 30 wt. % to about 70 wt. %, and in some embodiments, from about 40 wt. % to about 60 wt. % of the polymer matrix, and the second copolymer may likewise constitute from about 20 wt. % to about 80 wt. %, in some embodiments from about 30 wt. % to about 70 wt. %, and in some embodiments, from about 40 wt. % to about 60 wt. % of the polymer matrix.

In certain cases, ethylene vinyl acetate copolymer(s) constitute the entire polymer content of the core polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix.

One or more therapeutic agents (e.g., antipsychotics) are also dispersed within the core polymer matrix that are capable of prohibiting and/or treating a condition, disease, and/or cosmetic state a patient. The therapeutic agent may be prophylactically, therapeutically, and/or cosmetically active, systemically or locally. The therapeutic agent can be homogenously dispersed within the core polymer matrix. Typically, therapeutic agents will constitute from about 5 wt. % to about 60 wt. %, in some embodiments from about 10 wt. % to about 50 wt. %, and in some embodiments, from about 15 wt. % to about 45 wt. % of the core, while the core polymer matrix constitutes from about 40 wt. % to about 95 wt. %, in some embodiments from about 50 wt. % to about 90 wt. %, and in some embodiments, from about 50 wt. % to about 70 wt. % of the core. Suitable therapeutic agents will be further discussed hereinbelow.

The core may also optionally contain one or more excipients if so desired, such as radiocontrast agents, release modifiers, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the core. In one embodiment, for instance, a radiocontrast agent may be employed to help ensure that the device can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc. The core can also be formulated to have a desired flexural modulus of elasticity ranging from about 2 MPa to about 200 MPa.

To help further control the release rate from the implantable medical device, a hydrophilic compound may also be incorporated into the core that is soluble and/or swellable in water. When employed, the weight ratio of the ethylene vinyl acetate copolymer(s) the hydrophilic compounds within the core may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Regardless of the particular components employed, the core may be formed through a variety of known techniques, such as by hot-melt extrusion, injection molding, solvent casting, dip coating, spray coating, microextrusion, coacervation, compression molding (e.g., vacuum compression molding), etc. In one embodiment, a hot-melt extrusion technique may be employed. Hot-melt extrusion is generally a solvent-free process in which the components of the core (e.g., hydrophobic polymer, therapeutic agent(s), optional excipients, etc.) may be melt blended and optionally shaped in a continuous manufacturing process to enable consistent output quality at high throughput rates. This technique is particularly well suited to various types of hydrophobic polymers, such as olefin copolymers. Namely, such copolymers typically exhibit a relatively high degree of long-chain branching with a broad molecular weight distribution. This combination of traits can lead to shear thinning of the copolymer during the extrusion process, which help facilitates hot-melt extrusion. Furthermore, the polar comonomer units (e.g., vinyl acetate) can serve as an “internal” plasticizer by inhibiting crystallization of the polyethylene chain segments. This may lead to a lower melting point of the olefin copolymer, which improves the overall flexibility of the resulting material and enhances its ability to be formed into devices of a wide variety of shapes and sizes.

During a hot-melt extrusion process, melt blending may occur at a temperature range of from about 20° C. to about 200° C., in some embodiments, from about 30° C. to about 150° C., in some embodiments from about 40° C. to about 100° C., and in some embodiments, in some embodiments from about 100° C. to about 120° C., to form a polymer composition. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical and it may contain any number and/or orientation of threads and channels as is known in the art. For example, the screw typically contains a thread that forms a generally helical channel radially extending around a core of the screw. A feed section and melt section may be defined along the length of the screw. The feed section is the input portion of the barrel where the olefin copolymer(s) and/or therapeutic agent(s) are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4 Q/πR³, where Q is the volumetric flow rate (“m³/s”) of the polymer melt and R is the radius (“m”) of the capillary (e.g., extruder die) through which the melted polymer flows.

Once melt blended together, the resulting polymer composition may be in the form of pellets, sheets, fibers, filaments, etc., which may be shaped into the core using a variety of known shaping techniques, such as injection molding, compression molding, nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present disclosure. In one embodiment, an injection molding apparatus may be employed that includes a first mold base and a second mold base, which together define a mold cavity having the shape of the core. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape of the core (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 30° C. to about 120° C., in some embodiments from about 60° C. to about 110° C., and in some embodiments, from about 30° C. to about 60° C.

Specifically, in embodiments, forming the implantable device includes melt-blending the core polymer matrix and one or more therapeutic agents in an extruder barrel at a first temperature and then mixing the core polymer matrix and therapeutic agent at a second temperature in the extruder barrel before extruding the mixture to form a core for the implantable device. The temperature during melt-blending can be the same or different from the temperature during mixing. Suitable temperatures range from about 70° C. to about 95° C., such as about 90° C. to about 95° C. Once extruded, the core can be cooled and then cut into suitable shapes to form the implantable device.

As indicated above, another suitable technique for forming a core of the desired shape and size is three-dimensional printing. During this process, the polymer composition may be incorporated into a printer cartridge that is readily adapted for use with a printer system. The printer cartridge may, for example, contain a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present disclosure. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al., which is incorporated herein in its entirety by reference thereto.

### II. Therapeutic Agents

A. Antipsychotics

As indicated above, therapeutic agents in the implantable device include one or more antipsychotics dispersed within the core and/or membrane layer(s). Antipsychotics generally refer to a class of therapeutic agents primarily used to manage and treat psychosis, such as schizophrenia. Antipsychotics are also used to treat bipolar disorder and major depressive disorder. Specifically, typical and some atypical antipsychotics are dopamine antagonists and act to impede dopamine in the brain. Further, atypical antipsychotics also influence serotonin.

Exemplary antipsychotics include both typical and atypical antipsychotics. Atypical antipsychotics that can be used herein include, but are not limited to, aripiprazole, clozapine, ziprasidone, paliperidone, risperidone, quetiapine, olanzapine, asenapine, iloperidone, lurasidone, brexpiprazole, cariprazine, and lumateperone. Salts, esters and/or isomers of antipsychotics are all meant to be encompassed in the scope of the present disclosure and shall be understood to fall under the term “antipsychotic”.

In certain embodiments, the therapeutic agent includes risperidone. Risperidone is an atypical antipsychotic and is indicated for the treatment of schizophrenia, irritability associated with autistic disorder, and as monotherapy or adjunctive therapy with lithium or valproate for the treatment of acute manic or mixed episodes associated with Bipolar 1 Disorder. Risperidone belongs to the chemical class of benzisoxazole derivatives. Risperidone has a molecular weight of 410.49 and a molecular formula of C₂₃H₂₇FN₄O₂. The structural formula of risperidone is shown below.

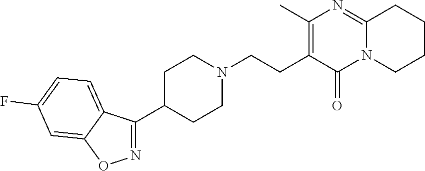 [SMILES: Cc1nc2n(c(=O)c1CCN1CCC(c3noc4cc(F)ccc34)CC1)CCCC2]

Risperidone is a monoaminergic antagonist with high affinity for the serotonin Type 2 (5HT₂), dopamine Type 2 (D₂), α1 and α2 adrenergic, and H₁ histaminergic receptors. Risperidone also shows low to moderate affinity for the serotonin 5HT_{1c}, 5HT_{1D}, 5HT_{1A} receptors and weak affinity for the dopamine D₁ and haloperidol-sensitive sigma site. Risperidone generally shows no affinity for cholinergic muscarinic or β₁ and β₂ adrenergic receptors.

B. Other Therapeutic Agents

Therapeutic agents utilized in the implantable device can further include other therapeutic agents, such as antidepressants, that are typically co-administered with antipsychotics. Additionally, other therapeutic agents can be administered with antipsychotics as disclosed herein in order to treat or prevent side effects from the antipsychotic medication.

### III. Membrane Layer(s)

As indicated above, the implantable device can optionally include one or more membrane layers (e.g., a first membrane layer) that is positioned adjacent to an outer surface of a core. Additional membrane layers (e.g., a second membrane layer, a third membrane layer, etc.) may be layered on the core as desired. The number of membrane layers may vary depending on the particular configuration of the device, the nature of the therapeutic agent, and the desired release profile. For example, in certain embodiments, the device may contain only one membrane layer.

When employed, the membrane polymer matrix contains at least one ethylene vinyl acetate copolymer, such as described in more detail above. The vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The melting temperature of the ethylene vinyl acetate copolymer may also range from about 40° C. to about 140° C., in some embodiments from about 50° C. to about 125° C., and in some embodiments, from about 60° C. to about 120° C., as determined in accordance with ASTM D3418-15. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55). In embodiments, the ethylene vinyl acetate copolymer in the membrane polymer matrix is from about 20 wt. % to about 90 wt. %, such as from about 30 wt. % to about 80 wt. %, such as from about 40 wt. % to about 70 wt. %.

In certain cases, ethylene vinyl acetate copolymer(s) constitute the entire polymer content of the membrane polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix. The membrane polymer matrix typically constitutes from about 50 wt. % to 99 wt. %, in some embodiments, from about 55 wt. % to about 98 wt. %, in some embodiments from about 60 wt. % to about 96 wt. %, and in some embodiments, from about 70 wt. % to about 95 wt. % of a membrane layer.

To help further control the release rate from the implantable medical device, a hydrophilic compound may also be incorporated into the membrane layer(s) that is soluble and/or swellable in water. When employed, the weight ratio of the ethylene vinyl acetate copolymer(s) the hydrophilic compounds within the membrane layer may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Optionally, the membrane layer(s) can include a plurality of water-soluble particles distributed within a membrane polymer matrix. The particle size of the water-soluble particles is controlled to help achieve the desired delivery rate. More particularly, the median diameter (D50) of the particles is about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles are also selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt. % to about 50 wt. %, in some embodiments from about 2 wt. % to about 45 wt. %, in some embodiments from about 4 wt. % to about 40 wt. %, and in some embodiments, from about 5 wt. % to about 30 wt. % of a membrane layer.

When employing multiple membrane layers, it is typically desired that each membrane layer contains a polymer matrix includes an ethylene vinyl acetate copolymer. Additionally, each of the membrane layers can include a plurality of water-soluble particles distributed within a membrane polymer matrix that includes an ethylene vinyl acetate copolymer. For example, a first membrane layer may contain first water-soluble particles distributed within a first membrane polymer matrix and a second membrane layer may contain second water-soluble particles distributed within a second membrane polymer matrix. In such embodiments, the first and second polymer matrices may each contain an ethylene vinyl acetate copolymer. The water-soluble particles and ethylene vinyl acetate copolymer(s) within one membrane layer may be the same or different than those employed in another membrane layer. In one embodiment, for instance, both the first and second membrane polymer matrices employ the same ethylene vinyl acetate copolymer(s) and the water-soluble particles within each layer have the same particle size and/or are formed from the same material. Likewise, the ethylene vinyl acetate copolymer(s) used in the membrane layer(s) may also be the same or different the hydrophobic polymer(s) employed in the core. In one embodiment, for instance, both the core and the membrane layer(s) employ the same ethylene vinyl acetate copolymer. In yet other embodiments, the membrane layer(s) may employ an ethylene vinyl acetate copolymer that has a lower melt flow index than a hydrophobic polymer employed in the core. Among other things, this can further help control the release of the therapeutic agent from the device. For example, the ratio of the melt flow index of a hydrophobic polymer employed in the core to the melt flow index of an ethylene vinyl acetate copolymer employed in the membrane layer(s) may be from about 1 to about 20, in some embodiments about 2 to about 15, and in some embodiments, from about 4 to about 12.

If desired, membrane layer(s) used in the device may optionally contain a therapeutic agent, such as described below, which is also dispersed within the membrane polymer matrix. The therapeutic agent in the membrane layer(s) may be the same or different than the therapeutic agent employed in the core. When such a therapeutic agent is employed in a membrane layer, the membrane layer generally contains the therapeutic agent in an amount such that the ratio of the concentration (wt. %) of the therapeutic agent in the core to the concentration (wt. %) of the therapeutic agent in the membrane layer is greater than 1, in some embodiments about 1.5 or more, and in some embodiments, from about 1.8 to about 4. When employed, therapeutic agents typically constitute only about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of therapeutic agents prior to release from the core. When multiple membrane layers are employed, each membrane layer may generally contain the therapeutic agent in an amount such that the ratio of the weight percentage of the therapeutic agent in the core to the weight percentage of the therapeutic agent in the membrane layer is greater than 1, in some embodiments about 1.5 or more, and in some embodiments, from about 1.8 to about 4.

The membrane layer(s) may also optionally contain one or more excipients as described above, such as radiocontrast agents, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

The membrane layer(s) may be formed using the same or a different technique than used to form the core, such as by hot-melt extrusion, compression molding (e.g., vacuum compression molding), injection molding, solvent casting, dip coating, spray coating, microextrusion, coacervation, etc. In one embodiment, a hot-melt extrusion technique may be employed. The core and membrane layer(s) may also be formed separately or simultaneously. In one embodiment, for instance, the core and membrane layer(s) are separately formed and then combined together using a known bonding technique, such as by stamping, hot sealing, adhesive bonding, etc. Compression molding (e.g., vacuum compression molding) may also be employed to form the implantable device. As described above, the core and membrane layer(s) may be each individually formed by heating and compressing the respective polymer compression into the desired shape while under vacuum. Once formed, the core and membrane layer(s) may be stacked together to form a multi-layer precursor and thereafter and compression molded in the manner as described above to form the resulting implantable device.

### IV. Device Configurations

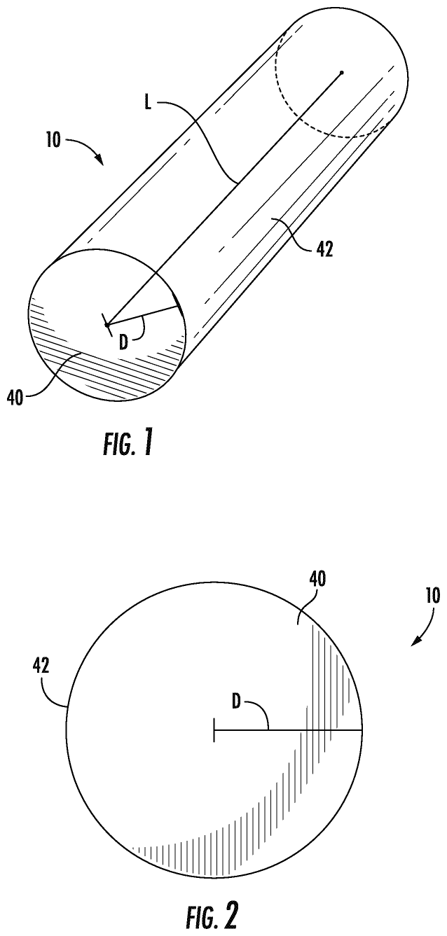

Referring to FIGS. 1-2, for example, one embodiment of an implantable device 10 is shown. The implantable device 10 includes a core 40 having a generally circular cross-sectional shape and is elongated so that the resulting device is generally cylindrical in nature. During use of the device 10, a therapeutic agent is capable of being released from the core 40 so that it exits from the outer surface 42 of the implantable device 10.

As shown, the implantable device can have a length (L) and a cross-sectional diameter (D). The length (L) can range from about 2.5 cm to about 7 cm, such as about 3 cm to about 6 cm, such as about 4 cm to about 5 cm. In certain embodiments, the length (L) is about 5 cm. The cross-sectional diameter (D) can range from about 2 mm to about 5 mm, such as from about 3 mm to about 4 mm. In embodiments, the cross-sectional diameter is about 3.5 mm. The device can be sized according to desired therapeutic agent loading and implantation time. For example, for longer lasting implants, the size can be increased such that the implant can be loaded with enough therapeutic agent to las for the life of the implant.

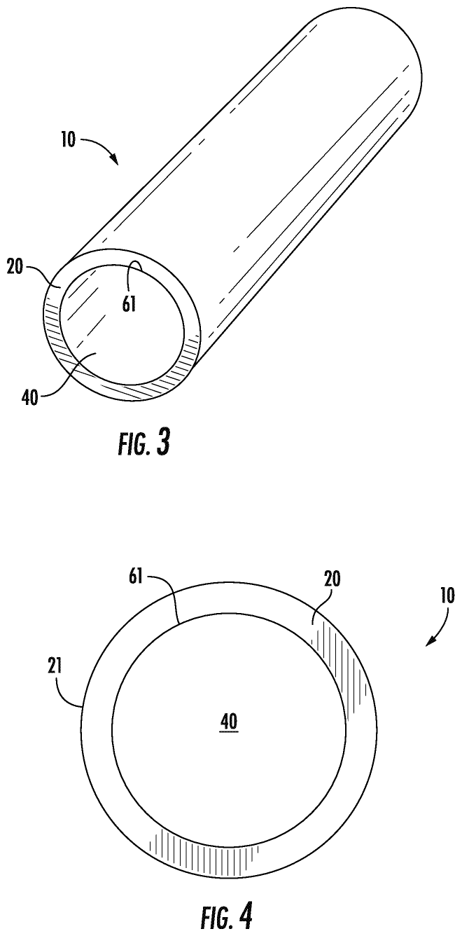

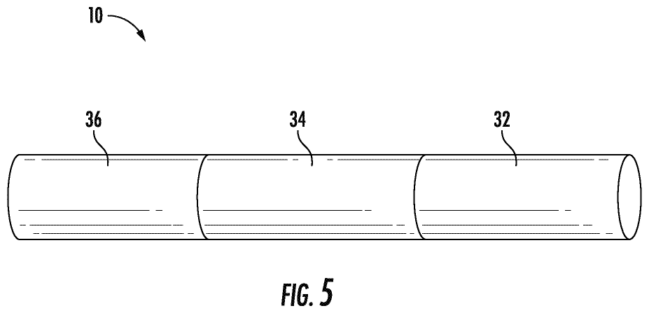

Another embodiment of an implantable device 10 is shown in FIG. 3-4. The core 40 has a generally circular cross-sectional shape and is elongated so that the resulting device is generally cylindrical in nature. The core 40 defines an outer circumferential surface 61 about which a membrane layer 20 is circumferentially disposed. Similar to the core 40, the membrane layer 20 also has a generally circular cross-sectional shape and is elongated so that it covers the entire length of the core 40. During use of the device 10, a therapeutic agent is capable of being released from the core 40 and through the membrane layer 20 so that it exits from an external surface 21 of the device.

Of course, in other embodiments, the device may contain multiple membrane layers. In the device of FIGS. 3-4, for example, one or more additional membrane layers (not shown) may be disposed over the membrane layer 20 to help further control release of the therapeutic agent. In other embodiments, the device may be configured so that the core is positioned or sandwiched between separate membrane layers.

As shown in FIG. 5, the implantable device 10 can include one or more compartments. As shown, the device includes three compartments 32, 34, and 36, however, the disclosure is not so limited. Indeed, two-compartment devices are conceivable in accordance with present disclosure. In fact, any number of compartments or sections can be joined together to form an implantable device as provided herein. As shown, the implantable device 10 includes a first compartment 32, a second compartment 34, and a third compartment 36. Advantageously, the compartments 32, 34, and 36 can each be formulated to contain different amounts of therapeutic agents or different therapeutic agents depending on desired results as will be further discussed hereinbelow. It is also conceivable that the compartments 32, 24, and 36 can be formed from the same core polymer matrix or can each be formed from different core polymer matrix materials. For example, core polymer matrix materials can be modified such that the compartments can have different release rates for therapeutic agents contained therein. Furthermore, any suitable materials can be used or placed between compartments when molding the implantable device.

Additional membrane layers can be added to the implantable device of FIG. 5 as desired (not shown in FIG. 5). For example, in certain embodiments at least one membrane layer can surround the external surface of all compartments 32, 34, 36. In other embodiments, different membrane layers may surround different portions of the compartments 32, 34, and 36. For example, a first membrane can surround the first compartment 32, a second membrane can surround the second compartment 34, and a third membrane can surround the third compartment 36.

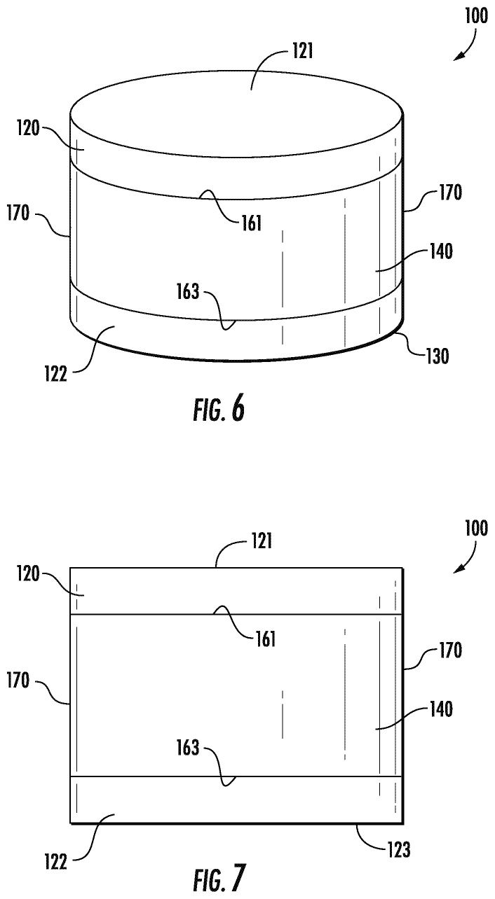

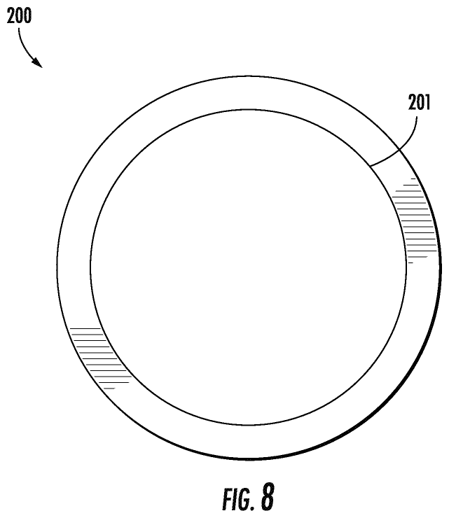

Referring now to FIGS. 6-7, for example, one embodiment of an implantable device 100 is shown that contains a core 140 having a generally circular cross-sectional shape and is elongated so that the resulting device is generally disc-shaped in nature. The core 140 defines an upper outer surface 161 on which is positioned a first membrane layer 120 and a lower outer surface 163 on which is positioned a second membrane layer 122. Similar to the core 140, the first membrane layer 120 and the second membrane layer 122 also have a generally circular cross-sectional shape that generally covers the core 140. If desired, edges of the membrane layers 120 and 122 may also extend beyond the periphery of the core 140 so that they can be sealed together to cover any exposed areas of an external circumferential surface 170 of the core 140. During use of the device 100, a therapeutic agent is capable of being released from the core 140 and through the first membrane layer 120 and second membrane layer 122 so that it exits from external surfaces 121 and 123 of the device. Of course, if desired, one or more additional membrane layers (not shown) may also be disposed over the first membrane layer 120 and/or second membrane layer 122 to help further control release of the therapeutic agent.

In embodiments, the implantable device can be a mono-lithic disc having no membranes or sheath layers disposed thereon. Further, the disc can have a thickness of about 1 mm to about 5 mm, such as about 2 mm to about 4 mm and a diameter of about 5 mm to about 35 mm, such as about 10 mm to about 30 mm, such as about 15 mm to about 25 mm, such as about 20 mm to about 30 mm. Increasing the dimensions of the disc can facilitate higher total loading amounts of one or more antipsychotics.

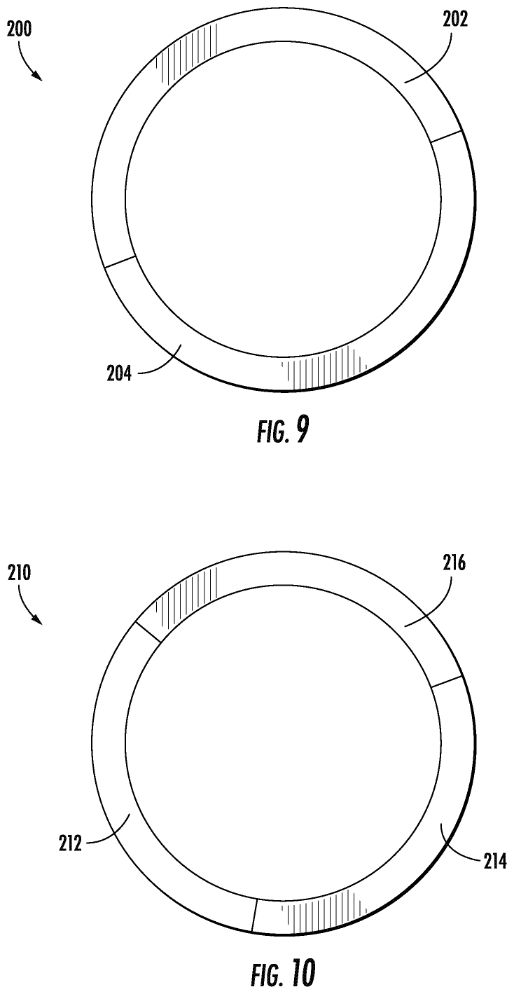

Referring now to FIGS. 8-10, in certain embodiments, the implantable device is a vaginal ring 200. FIG. 8 illustrates a single compartment 201 vaginal ring. However, in other embodiments, as shown in FIGS. 9-10, a multi-compartment ring can be formed. An example vaginal ring 200 is shown in FIG. 9 having at least two compartments 202, 204, while the ring 210 as shown in FIG. 10 includes at least three compartments 212, 214, 216. While two and three compartment examples are shown, the disclosure is not so limited. Indeed, the vaginal rings can include a plurality of compartments. In fact, any number of compartments or sections can be joined together to form a vaginal ring as provided herein. Furthermore, any suitable materials can be used or placed between compartments when molding the ring. Each compartment of the vaginal ring (e.g., 202, 204 or 212, 214, 216) can be the same or different. For example, for delivery of a combination of therapeutic agents the compartments can contain different types or amounts of therapeutic agents.

Multi-compartment cylinders or vaginal rings can be formed having different types and/or amounts of antipsychotics dispersed in each compartment. Such embodiments provide for the delivery of multiple antipsychotic compounds from the implantable device. In certain embodiments, the amount of antipsychotic delivered from each compartment can vary. Indeed, each compartment may be formulated with a different core polymer matrix and/or membrane layer in order to affect the release rate of antipsychotic from each compartment. For example, certain compartments can be configured to release antipsychotic faster in order to reach an initial steady state concentration, while the remaining compartments can be formulated to release antipsychotic more slowly such that sustained delivery of one or more antipsychotics over a period of time can be achieved.

The compartments disclosed herein can include one or more membrane layers as disclosed herein. The membrane layers of the compartments can be varied in order to further effect release of the dispersed therapeutic agents from the compartments.

### V. Use of Device

The implantable device of the present disclosure may be used in a variety of different ways to treat prohibit and/or treat a condition, disease, or cosmetic state in a patient. The term “implantable device” as used herein, is intended to cover a variety of implantable or insertable devices and associated methods of use. For example, the implantable device can be implanted into the body (e.g., subcutaneously) or the implantable device can be inserted into the body (e.g., intravaginally). The device may be implanted subcutaneously, orally, mucosally, etc., using standard techniques. The delivery route may be intrapulmonary, gastroenteral, subcutaneous, intramuscular, intravaginal, or for introduction into the central nervous system, intraperitoneum or for intraorgan delivery. As noted above, the implantable device may be particularly suitable for delivering a antipsychotic for treatment of schizophrenia, schizoaffective disorder, bipolar disorder, psychotic depression, major depressive disorder, and treatment resistant depression.

The implantable device can be in different forms, such as an implant (e.g., subcutaneous implant), an intrauterine system (IUS) (e.g., intrauterine device), a helical coil, a spring, a rod, a cylinder, and/or a vaginal ring. In embodiments, where the implantable device includes a vaginal ring, the core and any membrane layers of the ring can be formed as disclosed herein. For example, a method of manufacture of the ring-shaped device includes extrusion of the core containing the antipsychotic or co-extrusion of the core containing antipsychotic and one or more membrane layers, to render a rod or fiber. The rod/fiber can then be cut into pieces of required lengths and assembled into a ring-shaped device via any suitable molding procedure. For example, an implantable device in the form of a rod can be formed and the ends of the rod can be joined together to form a ring. Additional membrane layers, as required, can be incorporated and/or layered on the vaginal ring.

Through selective control over the particular nature of the device and the manner in which it is formed, the resulting device can be effective for sustained release of one or more antipsychotics over a prolonged period of time. For example, the implantable device can release the therapeutic agent(s) for a time period of about 5 days or more, in some embodiments about 10 days or more, in some embodiments from about 21 days or more, and in some embodiments, from about 25 days to about 50 days (e.g., about 30 days). In certain embodiments, the implantable device can release the therapeutic agent(s) for a time period for about 3 months or more, such as about 6 months or more, such as about 12 month or more, and in some embodiments, from about 12 months to about 36 months. Further, the therapeutic agent(s) can be released in a controlled manner (e.g., zero order or near zero order) over the course of the release time period. After a time period of 21 days, for example, the cumulative release ratio of the implantable medical device may be from about 20% to about 70%, in some embodiments from about 30% to about 65%, and in some embodiments, from about 40% to about 60%. Likewise, after a time period of 30 days, the cumulative release ratio of the implantable medical device may still be from about 40% to about 85%, in some embodiments from about 50% to about 80%, and in some embodiments, from about 60% to about 80%. The “cumulative release ratio” may be determined by dividing the amount of the therapeutic agent released at a particulate time interval by the total amount of therapeutic agent initially present, and then multiplying this number by 100.

Of course, the actual dosage level of the antipsychotic delivered will vary depending on the particular antipsychotic employed and the time period for which it is intended to be released. The dosage level is generally high enough to provide a therapeutically effective amount of the antipsychotic to render a desired therapeutic outcome, i.e., a level or amount effective to reduce or alleviate symptoms of the condition for which it is administered. The exact amount necessary will vary, depending on the subject being treated, the age and general condition of the subject to which the antipsychotic is to be delivered, the capacity of the subject's immune system, the degree of effect desired, the severity of the condition being treated, the particular antipsychotic selected and mode of administration of the composition, among other factors. An appropriate effective amount can be readily determined by one of skill in the art. For example, an effective amount will typically range from about 1.5 mg to about 3 mg per day, such as from about 2 mg to about 2.5 mg per day, such as about 1.5 mg of the antipsychotic delivered per day. In certain embodiments, an effective amount can range from about 3 mg to about 5 mg per day, such as from about 4 mg.

Depending on the route of administration for delivery of the implant, the amount of antipsychotic loaded into the implant can vary. For example, for certain implants configured to release antipsychotics for periods of time equal to or greater than 6 months (e.g., subcutaneous implants), the implant (e.g., the core) is loaded with from about 60 mg to about 300 mg of one or more antipsychotics, such as from about 75 mg to about 275 mg, such as from about 100 mg to about 225 mg, such as from about 125 mg to about 200 mg. Certain implants can also be loaded with from about 500 mg to about 1000 mg of one or more antipsychotics. For instance, the implant can be loaded with from about 600 mg to about 900 mg, such as from about 700 mg to about 800 mg. Additionally, the amount of antipsychotic loaded into the core can be modified (e.g., increased and/or decreased) depending on the amount of implantation time desired or route of implantation (e.g., subcutaneously vs. intravaginally).

Further, one or more implantable devices can be utilized in a patient in order to provide the effective amount. For instance, in certain embodiments a disc-shaped implant according to the disclosed dimensions herein can be loaded with at least 1,000 mg of the antipsychotic and further can be sized such that it is capable of releasing from about 1 mg per day up to about 5 mg per day. Advantageously, use of the disc-shaped implant as provided can utilize a single implantable device for release of an effective amount of one or more antipsychotics for at least 3 months, such as at least 6 months.

If desired, the device may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the device may be stored. For example, a single film may be folded at one or more points and sealed along its periphery to define the cavity within with the device is located. To use the device, the package may be opened, such as by breaking the seal, and the device may then be removed and implanted into a patient.

### Examples 1-4

Ateva® 4030AC was compounded with Risperidone via 11 mm twin-screw extruder. 10% Risperidone loading was selected for Risperidone as shown in Table 1. Further, Ateva® 2820A was compounded with Risperidone via 11 mm twin-screw extruder. Three different loading percentages 10 wt. %, 20 wt. % and 50 wt. % were selected for Risperidone as shown in Table 1. A total of four different formulations were prepared. The diameter of the compounded filaments were 3-5 mm and all samples were cut into 0.5 to 0.8 mm lengths to perform an in vitro drug release study.

TABLE 1

Diameter (2 mm)
Length (1 cm)

| Example | Ateva ® 4030AC (wt. %) | Risperidone (wt. %) |
|---|---|---|
| 1 | 90 | 10 |
| | Ateva ® 2820A (wt. %) | Risperidone (wt. %) |
| 2 | 90 | 10 |
| 3 | 60 | 40 |
| 4 | 50 | 50 |

The release study of Risperidone loaded EVA rods was performed in a shaking incubator maintained at 37° C. Phosphate buffer, 150 mM, pH 7.0 was used as release media. At regular intervals, buffer was exchanged with fresh buffer. Risperidone in the elution buffer was characterized using either HPLC or UV-Vis spectrophotometer.

FIG. 11 illustrates the percent release of risperidone for Example 1-4 as referenced above in Table 1. Table 2 below provides the data points for % risperidone release as shown in FIG. 11.

TABLE 2

| | | | | |
|---|---|---|---|---|
| | 4030AC/ | 2820A/ | 2820A/ | 2820A/ |
| | 10% API | 10% API | 40% API | 50% API |
| Time | Example 1 | Example 2 | Example 3 | Example 4 |

| | |
|---|---|
| (hr) | Percent (%) Risperidone Release |

| | | | | |
|---|---|---|---|---|
| 24 | 3.520 | 1.746 | 1.798 | 6.043 |
| 48 | 4.159 | 2.095 | 2.348 | 8.335 |
| 72 | 5.259 | 2.862 | 2.815 | 9.731 |
| 144 | 7.885 | 4.887 | 3.809 | 12.222 |
| 312 | 12.786 | 8.738 | 5.429 | 16.211 |
| 672 | 19.589 | 13.738 | 7.570 | 20.164 |
| 984 | 24.315 | 16.858 | 8.843 | 22.387 |

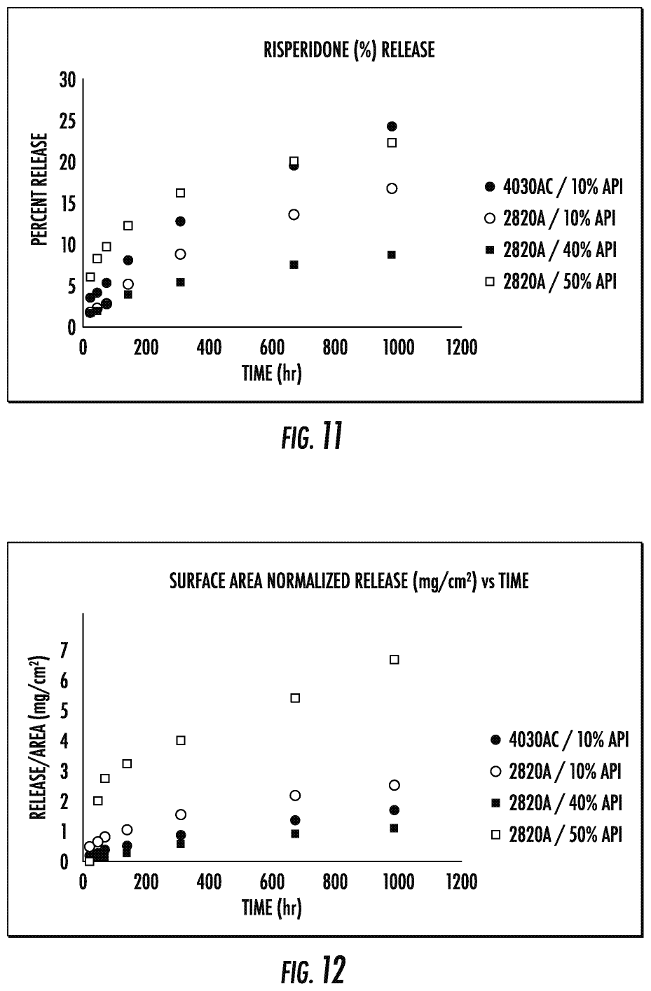

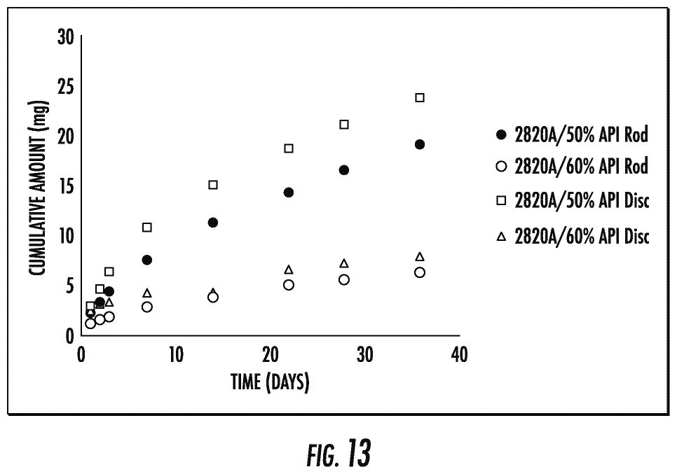

FIG. 12 illustrates the surface area normalized release of risperidone for Examples 1-4 as referenced above in Table 1. Table 3 below provides the data points for % risperidone release as shown in FIG. 12.

TABLE 3

| | | | | |
|---|---|---|---|---|
| | 4030AC/ | 2820A/ | 2820A/ | 2820A/ |
| | 10% API | 10% API | 40% API | 50% API |
| Time | Example 1 | Example 2 | Example 3 | Example 4 |

| | |
|---|---|
| (hr) | Surface area normalized release (mg/cm²) |

| | | | | |
|---|---|---|---|---|
| 24 | 0.243 | 0.118 | 0.510 | 1.995 |
| 48 | 0.288 | 0.141 | 0.667 | 2.750 |
| 72 | 0.366 | 0.193 | 0.801 | 3.211 |
| 144 | 0.550 | 0.329 | 1.084 | 4.033 |
| 312 | 0.892 | 0.589 | 1.545 | 5.351 |
| 672 | 1.366 | 0.925 | 2.155 | 6.655 |
| 984 | 1.699 | 1.135 | 2.518 | 7.389 |

### Example 5

Ateva® 2820A was compounded with risperidone via 11 mm twin-screw extruder. 60% w/w risperidone loading was selected as shown in Table 4. The diameter of the compounded filaments were 2-4 mm and the length of each was 1 cm to perform an in vitro drug release study.

TABLE 4

Diameter (2-4 mm)
Length (1 cm)

| Example | Ateva ® 2820A (wt. %) | Risperidone (wt. %) |
|---|---|---|
| 5 | 40 | 60 |

The release study of Risperidone loaded EVA rods was performed in a shaking incubator maintained at 37° C. Phosphate buffer, 150 mM, pH 7.0 was used as release media. At regular intervals, buffer was exchanged with fresh buffer. Risperidone in the elution buffer was characterized using either HPLC or UV-Vis spectrophotometer.

### Examples 6-7

Examples 6-7 were formed from Ateva®2820A having the following drug loadings 50 and 60% w/w risperidone. Examples 6-7 were further processed via vacuum compression molding (VCM) to form discs. Each of the discs had a diameter of about 2 cm and a thickness of 1-1.2 mm. Discs were formed to evaluate drug release from an implant having greater surface area as compared to the rods. Each disc was cut in half and the release study of the risperidone loaded EVA discs was performed in a shaking incubator maintained at 37° C. Phosphate buffer, 150 mM, pH 7.0 was used as release media. At regular intervals, buffer was exchanged with fresh buffer. Risperidone in the elution buffer was characterized using either HPLC or UV-Vis spectrophotometer.

FIG. 13 illustrates the percent release of risperidone for Examples 4-7 as referenced below in Table 5. Table 5 below provides the data points for % risperidone release as shown in FIG. 13.

TABLE 5

| | 50%2820A | 40%2820A | 50% 2820A | 40%2820A |
|---|---|---|---|---|
| | 50% API | 60% API | 50% API | 60% API |
| Time | Rod | Rod | Disc | Disc |
| (days) | Example 4 | Example 5 | Example 6 | Example 7 |
| 1 | 2.024 | 1.151 | 3.027 | 2.351 |
| 2 | 2.683 | 1.589 | 4.705 | 3.271 |
| 3 | 3.197 | 1.926 | 6.373 | 4.342 |
| 7 | 4.312 | 2.830 | 10.769 | 7.503 |
| 14 | 4.312 | 3.967 | 15.047 | 11.337 |
| 22 | 6.620 | 5.005 | 18.759 | 14.380 |
| 28 | 7.198 | 5.633 | 21.128 | 16.556 |
| 36 | 7.796 | 6.308 | 23.899 | 19.057 |

Table 6 illustrates the total drug loading for the disc implants of Examples 6-7.

TABLE 6
Total drug loading - Disc shaped implant
2 cm diameter and 1-1.12 mm thick

| | Weight of API in | Weight of API in | Average surface |
|---|---|---|---|
| | whole disc | half disc | area (cm²) |
| Example 6 | 184.45 | 92.225 | 6.796 |
| Example 7 | 233.4 | 116.7 | 6.726 |

Table 7 illustrates the total drug loading for the rod implants of Examples 4-5.

TABLE 7
Total drug loading - Rod shaped implant;
1 cm length and 2-4 mm diameter

| | Weight of | Average surface |
|---|---|---|
| | API in rod | area (cm²) |
| Example 4 | 68.617 | 1.714 |
| Example 5 | 26.710 | 0.818 |

Table 8 illustrates the ratio of total drug in the disc over rods for Examples 4-7. Briefly, to calculate the total drug loading ratio the weight amount of API in the disc (Examples 6-7) was divided by the weight amount of API in the rod (Examples 4-5) for the corresponding percentage loadings. As shown, given the dimensions of the disc, the discs were able to load at least twice as much weight of the API up to at least eight (8) times the amount of API.

TABLE 8

| | Ratio of total drug |
|---|---|
| | loading Disc/Rod |
| Examples 4 and 6 | 2.69 |
| Examples 5 and 7 | 8.74 |

As used herein, the use of the term “about” in conjunction with a numerical value is intended to refer to within twenty percent (20%) of the stated numerical value.

The methods, compositions, and devices of the present disclosure, including components thereof, can comprise, consist of, or consist essentially of the essential elements and limitations of the disclosure described herein. For instance, in embodiments the methods, compositions, and devices can be substantially free from one or more components disclosed herein. As used herein, the term “substantially free” means no more than an insignificant trace amount present and encompasses completely free (e.g., 0 molar % up to 0.01 molar %).

These and other modifications and variations of the present disclosure may be practiced by those of ordinary skill in the art, without departing from the spirit and scope of the present disclosure. In addition, it should be understood that aspects of the various embodiments may be interchanged both in whole or in part. Furthermore, those of ordinary skill in the art will appreciate that the foregoing description is by way of example only and is not intended to limit the disclosure so further described in such appended claims.

What is claimed is:

1. An implantable device for delivering one or more antipsychotics, the implantable device comprising:
a core comprising a core polymer matrix within which is dispersed a therapeutic agent comprising one or more antipsychotics, the core polymer matrix containing an ethylene vinyl acetate copolymer, wherein the ethylene vinyl acetate copolymer has a melt flow index of from about 1 to about 400 grams per 10 minutes as determined in accordance with ASTM D1238-20 at a temperature of 190° C. and a load of 2.16 kilograms.

2. The implantable device of claim 1, wherein the ethylene vinyl acetate copolymer has a melting temperature of from about 40° C. to about 120° C. as determined in accordance with ASTM D3418-15.

3. The implantable device of claim 1, wherein the core has a flexural modulus of elasticity of from about 2 to about 200.

4. The implantable device of claim 1, wherein the ethylene vinyl acetate copolymer has a vinyl acetate content of from about 10 wt. % to about 60 wt. %.

5. The implantable device of claim 1, wherein the core polymer matrix further includes one or more hydrophobic polymers.

6. The implantable device of claim 1, wherein the ethylene vinyl acetate copolymer in the core polymer matrix is from about 20 wt. % to about 90 wt. %.

7. The implantable device of claim 1, wherein the core polymer matrix includes a first ethylene vinyl acetate copolymer and a second ethylene vinyl acetate copolymer.

8. The implantable device of claim 1, wherein the one or more antipsychotics comprise risperidone, olanzapine, quetiapine, ziprasidone, aripiprazole, and functional analogues thereof.

9. The implantable device of claim 1, wherein the implantable device has a generally circular cross-sectional shape.

10. The implantable device of claim 1, wherein the device is in a form of a cylinder.

11. The implantable device of claim 10, wherein the cylinder includes one or more compartments.

12. The implantable device of claim 11, wherein the one or more compartments comprise a first compartment and a second compartment, wherein the therapeutic agent, an amount of therapeutic agent, or both in the first compartment is different from the therapeutic agent, an amount of therapeutic agent, or both in second compartment.

13. The implantable device of claim 12, further comprising a third compartment, wherein the therapeutic agent, an amount of therapeutic agent, or both in the third compartment is the same as either the first compartment or the second compartment, or is different from both the first compartment and the second compartment.

14. The implantable device of claim 10, wherein the cylinder has a length of about 2.5 cm to about 7 cm.

15. The implantable device of claim 10, wherein the cylinder has a diameter of about 2.0 mm to about 5 mm.

16. The implantable device of claim 1, wherein the device is in the form of a disc.

17. The implantable device of claim 16, wherein the disc has a diameter of from 5 mm to about 35 mm and a thickness of from about 1 mm to about 5 mm.

18. The implantable device of claim 1, wherein the device is in the form of a helical coil.

19. The implantable device of claim 1, wherein the device is in the form of a vaginal ring.

20. The implantable device of claim 19, wherein the vaginal ring includes one or more compartments.

21. The implantable device of claim 1, wherein the therapeutic agent in the core is from about 40 wt. % to about 80 wt. %.

22. The implantable device of claim 21, wherein the therapeutic agent in the core is about 60 wt. % to about 70 wt. %.

23. The implantable device of claim 1, wherein the core is loaded with from about 60 mg to about 300 mg of one or more antipsychotics.

24. The implantable device of claim 1, wherein the core is loaded with from about 600 mg to about 1,000 mg of one or more antipsychotics.

25. The implantable device of claim 1, wherein the device is capable of releasing the therapeutic agent for a time period of about 21 days or more.

26. The implantable device of claim 1, wherein the device is capable of releasing the therapeutic agent for a time period of about 3 months or more.

27. The implantable device of claim 1, wherein the device is capable of releasing the therapeutic agent for a time period of about 6 months or more.

28. The implantable device of claim 1, wherein the one or more antipsychotics are released from the device in an amount sufficient to deliver from about 0.5 mg of antipsychotic to about 5 mg of antipsychotic per day.

29. The implantable device of claim 28, wherein the one or more antipsychotics are released from the device in an amount sufficient to deliver from about 3 mg of antipsychotic to about 4 mg of antipsychotic per day.

30. The implantable device of claim 1, wherein the core polymer matrix comprises one or more hydrophilic compounds to control release of the therapeutic agent from the implantable device.

31. The implantable device of claim 30, wherein the one or more hydrophilic compounds are present in an amount of from about 1 wt. % to about 60 wt. %.

32. The implantable device of claim 1, wherein the therapeutic agent is homogenously dispersed within the core polymer matrix.

33. The implantable device of claim 1, wherein no membrane layers are present on the core.

34. The implantable device of claim 1, comprising one or more membrane layers.

35. The implantable device of claim 1, wherein the core is formed from a hot melt extrusion process.

36. The implantable device of claim 1, wherein the core is formed from compression molding.

37. A method for prohibiting and/or treating a condition, disease, and/or cosmetic state of a patient, the method comprising subcutaneously implanting one or more of the device of claim 1 in the patient.

38. A method for prohibiting and/or treating a condition, disease, and/or cosmetic state of a patient, the method comprising intravaginally inserting one or more of the device of claim 1 in the patient.

39. The method of claim 37, wherein the disease is schizophrenia.

40. A method for inhibiting D₂ dopaminergic receptors and 5-HT_{2A} serotonergic receptors in the brain, comprising subcutaneously inserting one or more implantable devices in a patient, the implantable device comprising a core comprising a core polymer matrix within which is dispersed a therapeutic agent comprising one or more antipsychotics, the core polymer matrix containing an ethylene vinyl acetate copolymer, wherein the ethylene vinyl acetate copolymer has a vinyl acetate content of from about 10 wt. % to about 60 wt. % and/or a melting temperature of from about 40° C. to about 120° C. as determined in accordance with ASTM D3418-15.

41. A method of manufacturing an implantable device, comprising:
melt-blending a core polymer matrix containing an ethylene vinyl acetate copolymer and a therapeutic agent comprising one or more antipsychotics in an extruder barrel at a first temperature, the ethylene vinyl acetate copolymer having a melt flow index of from about 1 to about 400 grams per 10 minutes as determined in accordance with ASTM D1238-20 at a temperature of 190° C. and a load of 2.16 kilograms;
mixing the core polymer matrix and therapeutic agent in the extruder barrel at a second temperature to form a mixture of core polymer matrix and therapeutic agent;
extruding the mixture of core polymer matrix and therapeutic agent from the extruder barrel forming a core of the implantable device;
cooling the core; and
cutting the core to form the implantable device.

42. The method of claim 41, wherein the first temperature is from about 70° C. to about 95° C.

43. The method of claim 41, wherein the second temperature is about 70° C. to about 95° C.

44. The method of claim 41, wherein the first temperature and the second temperature are the same.

45. The method of claim 41, wherein the first temperature and the second temperature are different.

46. The method of claim 41, wherein the ethylene vinyl acetate copolymer has a vinyl acetate monomer content of from about 25% to about 32% and the first temperature and second temperature range from about 90° C. to about 95° C.

47. The method of claim 46, wherein the core comprises from about 50 wt. % to about 70 wt. % of the one or more antipsychotics.

48. The method of claim 47, wherein the one or more antipsychotics comprise risperidone.

49. The method of claim 41, wherein the core is loaded with about 500 mg to about 1,000 mg of the one or more antipsychotics.
